(19) United States
(12) Patent Application Publication
(10) Pub. No.: US 2002/0042958 A1
(43) Pub. Date: Apr. 18, 2002

(54) System and method of formulating composition for color-treating hair

(75) Inventors: Cindy L. Orr, Port Washington, NY (US); Leland Hirsh, Roslyn Heights, NY (US); Andrew Bartfield, Williston Park, NY (US)

Correspondence Address: Robert E. Alderson, Jr., Esq., Kramer Levin Naftails & Frankel LLP, 919 Third Avenue, New York, NY, 10022, US

(73) Assignee: ARTEC SYSTEMS GROUP, INC.

(21) Appl. No.: 09/940,707

(22) Filed: Aug. 28, 2001

Related U.S. Application Data
(60) Provisional application No. 60/228,714, filed on Aug. 29, 2000.

(51) Int. Cl. A61K 7/13

(52) U.S. Cl. 8/405

(57) ABSTRACT

A system and method of on-site formulation of a personalized permanent haircolor product. The system and method are used to produce on-site at the salon, a permanent haircolor tailored to the color and the ratio of unpigmented hair to pigmented hair of each individual. The key to choosing the correct color for an individual is by predetermining the amount of un-pigmented to pigmented hair and selecting from a stock palette of formulas divided into two or more and preferably three series of tones varying in depth and tone depending on the ratio of un-pigmented to pigmented hair.

### CROSS-REFERENCE TO RELATED APPLICATION

The present application claims the benefit of priority from U.S. application Serial No. 60/228,714 filed Aug. 29, 2000.

### FIELD OF THE INVENTION

The present invention relates generally to personalized hair coloring compositions formulated based on the ratio of un-pigmented hair to pigmented hair of individuals; and more particularly, to a method and system used to produce on-site, e.g., at the salon, a personalized permanent haircolor in accordance with the original color and the ratio of un-pigmented hair to pigmented hair of the person for whom the hair color is produced. The key to choosing the correct mixture of stock formulations varying in depth and tone for an individual is predetermining the amount of un-pigmented to pigmented hair for that individual.

### BACKGROUND OF THE INVENTION

Inside the cortex of the hair fiber is the substance called melanin. Melanin is what gives the hair its natural color. It is the pigment of the hair. Two general classes of such pigments have been identified: eumelanins (characteristically brownish black) and pheomelanins (characteristically reddish orange). The concentration and combination of these two types of pigments are responsible for the natural color of hair. Dark hair has a higher concentration of the eumelanin, while red hair contains more pheomelanin. Light hair has reduced levels of both.

As hair ages, the pigments begin to break down under the natural influence of the environment, such as from exposure to sun or smoke and like pollutants. Also, in many individuals, the natural color of the hair may discolor with age or illness and develop an undesirable tone even without chemical processing or environmental exposure. As a result, hair may lose its natural brilliance over time. Additionally, in many people, hair follicles produce less and less pigment as a person gets older. As a result, new growth of hair may be gray or white. The graying of hair is typically a gradual process, and not all hair may turn gray at once.

Furthermore, the growing demand for new hairstyles has shaped a lucrative industry for salons and hair stylists. The ability to change the appearance of one's hair through chemical processes is important to both men and women. More specifically, it is important for both men and women to be able to chemically relax or to permanently wave the hair so that they may keep up with the rapidly changing hair style fashion in the hair industry. In some instances, such chemical processes, and alkali hair relaxers in particular, have the undesirable and very noticeable effect of discoloring of the natural underlying color of hair, particularly naturally blonde and gray hair.

For many people, the discoloration and graying of their hair is undesirable. As a result, many people seek to cover up or reduce the appearance of gray in their hair. These people will typically color their hair with a hair color formulation intended to deposit color on their hair. In many people, gray, or white un-pigmented hair is blended in with that person's naturally pigmented hair and it is impractical to deposit color only on the un-pigmented portions of their hair. As a result, these people will require a hair color formulation to both cover up the un-pigmented hair and add color to their pigmented hair.

Traditional haircolor systems and their method of use are based on levels (or depth) and tones. Typically, several stock formulations are provided that vary in the shade. A colorist in a salon, referring to memory or by comparing to a set of standard swatches, will determine the natural base level of the clients hair, determine the level they wish to achieve and determine the tone desired. The colorist will then chose the appropriate shade (or mix of shades) from the given stock of permanent color alternatives, or palette. The various shades in the stock palette of permanent haircolors are not formulated based on the amount of pigmented versus un-pigmented hair.

There exists a need, therefore, for a system and a method of preparing appropriate hair color formulations on-site tailored to the ratio of un-pigmented to pigmented hair of each customer's hair, which will produce a hair coloring formula precisely tailored to the customer's hair color, tone and proportion of gray, which eliminates the inventory and stock problems associated with the current practice, which will permit the colorist a high degree of creative control in addressing the gamut of hair tones and colorings which will be encountered in practice, and which will address the ad-hoc experimentation of the current practice to mix personalized hair color formulations from stock offerings.

There are known in the prior art various industry-available dye color mixing applications. These applications are typically directed to the formulation of the color dyes themselves for their use in the actual salon hair coloring process. For instance, a number of industry applications are directed to allowing a colorist to cross-reference a client's hair color level against either a hair tone or a desired color level, so as to permit the colorist to select a single pre-mixed color dye from a large quantity of different pre-mixed dyes that are shipped by the manufacturer of the system for use with the system. Typically, these dyes are then mixed with a so-called “generator” or “developer” to activate the color for the processing treatment. The colorist's choice of the single, appropriate pre-mixed dye product is sometimes governed by a chart which cross-references hair color level against hair tone or desired color level. The available pre-mixed dyes are listed at various locations on the chart, typically with a single, pre-mixed dye satisfying a small number of combinations, and often only one, of specific hair color level against desired hair color level or hair tone. Examples of such systems include the Logics International, Inc. “Color Facts” and “Attitudes” dye product-system; the Synaplex “Mid-Color” Ultra-Shine No-Lift Hair Color system; and the Wella “Color Charm” Liquid Cream Hair Color system.

Certain other color dye preparation systems provide the colorist with some degree of guidance to adapt certain particular pre-mixed dyes, or combinations thereof, so as to address a particular client's hair. These systems oftentimes extend to voluminous or otherwise cumbersome reference materials which must be utilized or cross-referenced in accordance with the base system. An example of such system is described in the Clairol Professional Encyclopedia, “Hair Color”. For instance, the Clairol Encyclopedia refers to a so-called “Torrids” formulator chart cross-referencing a natural hair color against a desired hair color. The chart features a series of individual discrete boxes, each box representative of a single natural hair color level and a single desired hair color level, leading the colorist to select one or more hair dyes which can be used independently or mixed to some degree to meet that single natural hair color level and that single desired hair color level. Separate from the chart, the Encyclopedia provides the colorist with certain textual recommendations for use with the Torrids system, in advising of a custom dye formulation table listing for the colorist certain customized formulas of two or more volumetric quantities of color dyes. However, these recommendations tend to be for specific coloring instances only and are not tied to any particular organized methodology to prepare color dye mixes for any type of possible hair coloring situation. Various aspects of color dye mixing is also referenced in the Schwarzkopf Color Technical Manual (albeit without the provision of a chart correlating hair colors and tones) and in the Framesi 2001 Hair Color Training Manual.

As will be appreciated, most of the existing color dye systems employ a large number of pre-mixed color dyes. Typically, it is the intent of these systems that the colorist select a single pre-mixed dye from a large number used in the particular coloring system. The intent of these color dye systems is to select a color dye which will change the color of a person's hair, cover gray hair, or the like. In effect, then, these systems endeavor to make a precise color selection for a very specific, narrow characteristic of a person's hair. While certain of the dye systems include some background information as to combining more than one pre-mixed dye to accommodate certain enumerated particular hair coloring needs, the information is oftentimes not made integral with the bulk of the system and is provided on an ad hoc basis of specific examples and with no formal organization. In fact, the modification information is typically embodied in a textual form separate and apart from the depictions used in the system, and the information is oftentimes limited to a discrete number of particular coloring applications, without any particular methodology or organization to guide the colorist to modifications across a wide gamut of potential hair coloring situations. Ultimately, other than the several specific examples given, these systems give little guidance to the colorist in formulating a personalized hair color composition for an individual. Generally, then, owing to the existing concerns, these systems are not adapted to on-site preparation of a hair color product based on the ratio of un-pigmented to pigmented hair.

What is desired is a method and system that allows the hair colorist to personalize the shade selection for a hair color formulation based on the client's amount of pigmented to un-pigmented hair. For the client, the resulting haircolor will look more natural and for the colorist, the selection and the amount of mixing of the color for their client is simplified and more precise. For the manufacturer, a limited number of shades can be organized into a system providing comprehensive coloring solutions covering the complete gamut of possible hair coloring requirements.

### SUMMARY OF THE INVENTION

The present invention provides a method of using a stock palette of permanent haircolor formulas consisting of different tones and levels. The shades in the palette have been formulated to create mixtures of the stock formulas based on the amount of pigmented versus un-pigmented hair. More particularly, in accordance with the invention, the palette of the stock permanent haircolor formulas is divided into at least two and preferably three different groups of tonal series. For example, “Group 1” or “naturals,” “Group 2” or “bright naturals,” and “Group 3” or “brights and cools.”

The advantage and the key to the use of the present invention allows the hair colorist to personalize the shade selection based on the client's amount of pigmented to un-pigmented hair. For the client, the resulting haircolor will look more natural and for the colorist, the selection and the amount of mixing of the color for their client is simplified.

Therefore, it is an object of the present invention to provide a system and method of formulating personalized hair coloring compositions based on the ratio of un-pigmented to pigmented hair of a person.

It is another object of the present invention to provide a palette of stock hair color formulas divided into two or more and preferably three groups of tonal series which are associated with the ratio of un-pigmented to pigmented hair.

It is a further object of the present invention to provide at least two and preferably three groups of tonal series of stock hair color formulas which may be used or mixed to formulate a personalized hair color composition based on the ratio of un-pigmented to pigmented hair of a person.

It is yet another object of the present invention to provide a guide to formulating a personalized hair color formulation in which the hair colorist determines the ratio of un-pigmented to pigmented hair of a person and selects and mixes hair color formulas from a palette divided into at least two and preferably three groups of tonal series to generate a personalized hair color composition for that person.

It is yet a further object of the present invention to provide a system and method for formulating hair color compositions for individual users which may be produced on-site in the salon from a limited stock palette, thereby obviating inventory requirements and the need for maintaining a multifarious stock of pre-prepared hair color compositions.

It is still another object of the present invention to provide a system and method for on-site formulation of hair color compositions for individual users to accommodate a wide variety of hair tones and colorings which can be easily modified by the colorist as desired, and which will yield a consistent product each time it is used that is appropriate for the individual's hair color and tone.

Still other objects and advantages of the invention will in part be obvious and will in part be apparent from the specification.

In accordance with the present invention, these and other objectives are achieved by a method and system of formulating personalized hair coloring compositions based on the ratio of un-pigmented hair to pigmented hair of a person; and more particularly, a method and system are used to produce on-site, e.g., at the salon, a personalized permanent haircolor in accordance with the original color and the ratio of un-pigmented hair to pigmented hair of the person for whom the hair color is produced. In a preferred embodiment, the palette of stock permanent haircolor formulas is divided into three different groups of tonal series. “Group 1” or “naturals,” “Group 2” or “bright naturals,” and “Group 3” or “brights and cools.” The key to choosing the correct mixture of stock formulations varying in depth and tone for an individual is predetermining the amount of un-pigmented to pigmented hair for that individual.

The invention accordingly comprises the several steps and the relation of one or more of such steps with respect to each of the others, and the composition embodying the several elements and the combination of one or more of such elements, all as exemplified in the following detailed disclosure.

### DETAILED DESCRIPTION OF THE PREFERRED EMBODIMENTS

The personalized permanent haircolor in accordance with the invention is formulated from a hair color stock palette comprising at least two and preferably three groups of tonal series which are selected and mixed in accordance with the ratio of un-pigmented hair to pigmented hair of the person for whom the formulation is made.

In general, each level of color in each series in the stock haircolor palette is formulated to have more depth than tone on un-pigmented hair, a balance of depth and tone for blended pigmented and un-pigmented hair, and more tone with less depth for pigmented hair.

Thus, for example, when coloring hair with over 50% un-pigmented hair, more brown or blonde is needed than tone to recreate the depth and richness back into the hair. Combination hair or blended gray hair (25-50% un-pigmented hair) needs a substantial amount of depth and tone. Finally, pigmented hair (less than 25% un-pigmented hair) needs to be lightened and given a balance of tone to “over-ride” the warmth of the pigmented hair. At the same time, a sufficient amount of depth must be present for coverage on the un-pigmented hair. Hair that is mostly pigmented (less than 25% un-pigmented) needs sufficient tone and lift with less depth for vibrant color results.

An extensive study on the effects of various haircolor formulations on pigmented and un-pigmented hair (grey/white) at 100%, 50%, and 25% blends on natural hair, led to the development of a hair color stock palette organized and arranged in accordance with the ratio of pigmented to un-pigmented hair. This stock palette may be used to formulate on-site a personalized permanent hair color that yields a stock color mixing system having predictable results.

For purposes of explanation, and without limiting the scope of the disclosure, a preferred embodiment is described having three groups of tonal series. It will be readily understood by those of ordinary skill in the art who read this disclosure, how to adapt the method and system described herein when using two groups of tonal series, or more than three groups of tonal series.

In an example, “Group 1” is formulated for up to 100% un-pigmented hair. The group consists of different tonal series at different shade levels. Examples of group 1 shades include: neutral browns and blondes, brown to blonde coppers with more brown than copper, brown to blonde gold with more brown than gold. All the shades within the group add depth and richness to un-pigmented hair.

“Group 2” consists of rich, brilliant colors for pigmented hair with up to 50% blended un-pigmented hair. Combination or blended pigmented/un-pigmented hair needs substantial depth and more tone to “over-ride” the warmth of the pigmented hair. When used on over 50% blended un-pigmented hair, brighter colors are achieved with Group 2. Examples of group 2 shades include: golden browns to blondes with more gold than brown, copper browns to blondes with more copper than brown.

“Group 3” consists of very bright shades formulated for less than 25% blended un-pigmented hair. All the shades within the group add brightness and tone to pigmented hair.

Thus, an exemplary hair color stock palette will include stock formulas varying the relative amounts of depth and tone in each of the groups:

Group 1, or Naturals—more depth, less bright tone (for coverage up to 100% gray/white).

The naturals series is formulated with more depth (brown/blonde), and less bright tone (gold, copper, red, red violet) for rich coverage on hair up to 100% gray. It has the same tones as the corresponding bright naturals and brights but with more depth and less bright tone. The naturals restore depth & richness into the un-pigmented hair.

The naturals tones series include:

(N) neutrals,

(BG) brown/blonde gold,

(BC) brown/blonde copper,

(BR) brown red, and

(BRV) brown red violet.

Group 2, or Bright naturals—balanced depth and bright tones (for pigmented hair up to 50% blended gray/white).

When used on over 50% blended gray/white hair, brighter colors are achieved. The bright naturals have a balance of depth (brown/blonde) and bright tones (gold, copper, gold copper, red, red copper, & red violet). It provides a balance of depth to override the tone of pigmented hair, with sufficient depth for gray coverage on the un-pigmented hair.

The bright naturals tones series include:

(GB) gold brown/blonde,

(GCB) gold copper brown/blonde,

(CB) copper brown/blonde,

(RCB) red copper brown/blonde,

(RB) red brown, and

(RVB) red violet brown.

Group 3, or Brights—pure, bright tones (for pigmented hair up to 25% blended gray/white).

When used on hair over 25% blended gray/white, extremely bright colors are achieved. The brights series have the same tones as the corresponding naturals & bright naturals with pure bright tone (gold copper, copper copper, red copper, red, red red).

The brights tones series includes:

(GC) gold copper,

(C) copper,

(CC) copper copper,

(RC) red copper,

(R) red, and

(RR) red red.

An additional series, which may also be considered a part of the brights series, may be included which adds to the flexibility of the personalized permanent hair color system.

The Cools series—pure gray tones (for pigmented hair with up to 25% blended grey/white).

The cools have a pure gray tone for eliminating warmth (brassiness) on pigmented hair with up to 25% un-pigmented (gray/white) hair. The tones correspond with the neutrals, and override the unwanted warmth that typically occurs when coloring the hair.

The cools tone include:

(A) ash brown/blonde.

Of course, it will readily be appreciated that other divisions and naming conventions can be generated that provide the same benefit in allowing the selection and mixing of the various stock formulas to formulate an individualized hair color composition based on the ratio of pigmented to un-pigmented hair.

In a typical case, a single “base” composition is used and individual stock formulas made by adjusting the dye levels to make the appropriate shades for each group.

Examples of Such Formulations:

Example 1 will produce a brown copper shade appropriate for 100% un-pigmented or completely gray or white hair. Example 2 will produce a copper brown shade appropriate for 50% un-pigmented or combination hair. And, Example 3 will produce a bright copper shade appropriate for pigmented hair. By altering the levels of the dye intermediates in a formula, the shade will change. An experienced formulator will understand the adjustments in the dye levels and how they will affect the resulting color.

| Example | Ingredient | Percentage (w/w) |
|---|---|---|
| Example 1. | Hair color base (may consist of water, solvents such as | 97.498% |
| Lightest brown | isopropyl alcohol, surfactants, chelating agents, anti- | |
| copper. | oxidants, fatty alcohols, emulsifiers, viscosity | |
| | builders) | |
| | p-Phenylenediamine | 0.632% |
| | p-Aminophenol | 0.550% |
| | 2-Methylresorcinol | 0.496% |
| | Resorcinol | 0.468% |
| | 4-Amino-2-Hydroxytoluene | 0.236% |
| | m-Aminophenol | 0.120% |
| Example 2. | Hair color base (may consist of water, solvents such as | 97.214% |
| Lightest | isopropyl alcohol, surfactants, chelating agents, anti- | |
| Copper Brown | oxidants, fatty alcohols, emulsifiers, viscosity | |
| | builders) | |
| | p-Phenylenediamine | 0.528% |
| | p-Aminophenol | 0.780% |
| | 2-Methylresorcinol | 0.497% |
| | Resorcinol | 0.487% |
| | 4-Amino-2-Hydroxytoluene | 0.387% |
| | m-Aminophenol | 0.107% |
| Example 3. | Hair color base (may consist of water, solvents such as | 98.271% |
| Bright Copper | isopropyl alcohol, surfactants, chelating agents, anti- | |
| | oxidants, fatty alcohols, emulsifiers, viscosity | |
| | builders) | |
| | p-Phenylenediamine | 0.00% |
| | p-Aminophenol | 0.791% |
| | 2-Methylresorcinol | 0.00% |
| | Resorcinol | 0.00% |
| | 4-Amino-2-Hydroxytoluene | 0.938% |
| | m-Aminophenol | 0.00% |

Combination Color Tones

A hair color stock palette must be limited to a practical number of colors in each series. Yet, the colorist may wish to apply a color in between two of the available colors in the series. For example, in the bright naturals and the brights there are “combination” colors that are a balanced mixture of the color above and the color below in each series. Thus, RCB (red copper brown/blonde) is a combination of RB (red brown/blonde) and CB (copper brown/blonde).

The Levels System

In addition, the hair color stock palette includes different levels of color (which may be moderated in accordance with a developer system as described below):

1. black

2. dark brown

3. medium brown

4. light brown

5. lightest brown

6. dark blonde

7. medium blonde

8. light blonde

9. lightest blonde

10. extra light blonde

12 Super light blonde

In a preferred embodiment, the haircolor stock palette is a calibrated level system such that the distance between each level is evenly balanced. In such a system, fewer stock formulas are needed. Thus, when mixing a higher and lower level together in equal amounts, the colorist creates the level in the middle. For example, mixing equal amounts of 8GB with 6GB, yields 7GB, and mixing equal amounts of 10A and 8A gives you 9A.

Determining the Natural Level

The colorist uses conventional means to determine the percentage of pigmented versus un-pigmented hair at 25%, 50%, 75% & 100% blends of gray/white. Typically, the colorist may compare the hair to known hair samples or depictions. For example, the “blend finder” or top center panel in the ARTec Systems Group, Inc. enamels® Consultation Center, provides a scale to measure against.

The neutral series represents natural pigmented hair. Use these swatches or depictions to determine the natural level.

Thus, for example, in accordance with a preferred embodiment of the invention, a hair color stock palette is provided as a series of 3 oz. tubes of hair color. Each 3 ounce tube of enamels permanent creme gel haircolor is identified by a number indicating the level in accordance with the above level designations and letter(s) indicating the tone in accordance with the above tone designations. In this case, “B” always represents brown or blonde.

For example: 3BR designates medium brown red.

A 3 oz. tube should be sufficient to provide at least two haircolor applications, and will preferably be marked in ½ ounce increments.

In a preferred embodiment, the hair color will be in the form of a creme gel having a viscosity that allows for brush/bowl or bottle application.

In a preferred embodiment, Color tone tabs and labels specify the color tone of each series. The same color tone tabs appear on the swatches in the explanatory materials and as labels on the packaging materials for easy identification.

As is well understood by those of ordinary skill in the art, haircolor compositions work in combination with a developer. Preferably, the combination of haircolor and developer will provide:

superior conditioning benefits,

a creamy spreadable consistency,

a stay-put moist consistency (not too thin, not too thick) for client comfort, and

an appropriate viscosity for optional, convertible bowl and brush or bottle application.

In a preferred embodiment, the developer also comprises several formulations to finely and precisely control the lifting and depositing action in the haircolor formulations. Further, these several formulations should be completely intermixable, providing even greater flexibility.

For example, in a preferred embodiment, the developer is designated in accordance with the level of lift, for example, developer may be designated as 10, 20, 30, and 40 formulas.

10 Volume Developer

Formulated to reduce the lightening action of the permanent haircolor. 10 developer is used whenever minimized lifting action and increased color depth is desired.

20 Volume Developer

Formulated as the standard developer for the permanent haircolor. 20 developer is formulated to provide complete gray/white coverage and is used when standard lightening action and optimum color deposit and intensity are desired.

30 Volume Developer

Formulated to increase the lightening action of permanent haircolor. 30 developer is use when additional lightening action and optimum color deposit is desired.

40 Volume Developer

Formulated for maximum lightening action of permanent haircolor.

In a preferred example, the developers will vary in strength and concentration of hydrogen peroxide. Thus, 10 developer is 3% (by vol.) hydrogen peroxide, 20 developer is 6% (by vol.) hydrogen peroxide, 30 developer is 9% (by vol.) hydrogen peroxide, and 40 developer is 12% (by vol.) hydrogen peroxide.

The system and method of the present invention allows a colorist to formulate a personalized permanent hair color based on the ratio of pigmented hair to un-pigmented hair for the person for whom the formulation is prepared. In use, the colorist first determines the percentage of pigmented versus blended un-pigmented hair and the natural haircolor level of the person whose hair is to be colored.

For example, a blend finder may be used to determine the percentage of pigmented versus blended un-pigmented hair from 25%, 50%, 75% and 100%.

The neutral series as described above may be used to represent natural pigmented hair. Therefore, the colorist may use these color samples to determine the natural level of the client's pigmented hair.

A good colorist will also consider the effects of porosity and texture of the client's hair.

Next, the colorist determines the level of color appropriate for that client. For example, the colorist will decide whether the client's hair is to be lighter or darker or the same level. The colorist may then decide whether to change tone and whether to cover or blend gray.

Next, the colorist determines the desired haircolor tone to be applied. This is typically done by visually matching the color of the individual's hair to swatches provided in a kit along with an instruction manual explaining how to select the right stock formula and developer solution to achieve the desired result. The colorist thus can easily determine the specific tone and select the haircolor stock formula or mixture to use.

Finally, the colorist determines if he or she is lifting and depositing or attempting to get maximum deposit of color. The colorist thus selects the appropriate developer from maximum deposit or 1 level of lift using 10 volume developer, or standard or 2 levels of lift using 20 volume developer, or 3 levels of lift using 30 volume developer, and 4 levels of lift using 40 volume developer.

In general, the colorist will mix equal amounts (i.e. a 1:1 ratio) of developer with haircolor using a brush/bowl or bottle application. However, for super light blonde colors, the colorist will mix double the amount (i.e. a 2:1 ratio) of 40 volume developer with level 12 super light blondes using a brush/bowl or bottle application.

Generally, an application time of 30 to 45 minutes is appropriate, however, for super light blonde, a time of 45-50 minutes is appropriate.

The process of applying the hair color/developer formulation amount the described.

In applying the haircolor/developer formulation for “virgin” applications, i.e., to go lighter:

For Brights: Choose the brights in the desired level and tone. For the first ½ inch of hair from the scalp out (lower shaft) use the corresponding bright naturals. This will prevent the lower shaft from going too bright (hot re-growth). On the lengths of the hair use the brights in the desired level and tone.

For example: use 4RB ½ inch closest to the scalp and 4R through the lengths of the hair.

For Bright Naturals: Choose the bright naturals in the desired level and tone. For the first ½ inch of hair from the scalp out (lower shaft) use the corresponding naturals. This will prevent the lower shaft from going too bright. On the lengths of the hair use the bright naturals in the desired level and tone.

For example: use 4BR on the ½ inch closest to the scalp and 4RB on to the lengths of the hair.

For Naturals: Apply desired formula from ½ inch away from the scalp out. Process 20 minutes. Remix the same formula and apply on ½ inch section of hair closest to the scalp. Process until even (up to 45 minutes).

Apply formula to the first ½ inch section from the scalp.

These rules will generally apply:

Take ¼ inch partings.

Mix formula for mid-shaft and apply.

Process 30 to 45 minutes as appropriate.

In applying the haircolor/developer formulation to go darker:

Apply color from scalp to ends and process (up to 45 minutes).

On overly porous ends, do not immediately work through ends. Wait until hair is almost completely processed to avoid ends taking on too much tone.

In applying retouch application with hair color:

Apply to new growth only.

If necessary, when the processing time is almost through, replenish the previously colored hair.

A preferred embodiment of using the invention will include the following techniques:

In coloring hair with small or large patches of unpigmented hair (regrowth or virgin), using the bright naturals, apply the color on the entire head except the patches of gray. On the patches, use the naturals in the corresponding level and tone as the bright naturals. Using the brights, apply color to everything except the patches of gray. On the patches, use the bright naturals in the corresponding level and tone as the brights. Using the cools, apply color to everything except the patches of grey. On the patches, use the neutrals in the corresponding level.

In retouching un-pigmented hair, for example, retouching up to 100% un-pigmented hair with the naturals, the corresponding bright naturals in the same level and tone can be used on the ends to achieve more vibrancy. In retouching up to 50% un-pigmented hair colored with the bright naturals, the corresponding brights in the same level and tone can be used on the ends to achieve more vibrancy.

In creating different levels, mix a higher and lower level together to create the level in the middle. For example: mixing equal amounts of 8GB and 6GB yields 7GB.

In testing the color to be used, it is appropriate to use the color on a limited amount of hair, or conduct a strand test. Mix ⅛ oz. of the exact formula to be used plus ⅛ oz. developer. Apply from scalp to ends on a strand large enough to view results. Time for 30 minutes and rinse hair thoroughly and dry to evaluate the resultant color.

In accordance with the invention, we have disclosed novel personalized hair coloring compositions and methods of formulating that use a stock palette of permanent haircolor formulas consisting of different tones and levels. The shades in the palette are formulated to create mixtures of stock based on the amount of pigmented versus un-pigmented hair. The palette of the stock permanent haircolor formulas is divided into three 30 different groups of tonal series. “Group 1” or “naturals,” “Group 2” or “bright naturals,” and “Group 3” or “brights and cools.” The advantage and the key to the use of the system is that the hair colorist personalizes the shade selection based on the client's amount of pigmented to un-pigmented hair. For the client, the resulting haircolor will look more natural and for the colorist, the selection and the amount of mixing of the color for their client is simplified and more precise. The advantage of our method and system is that simplifies the selection of the proper shade for an individual by the hair colorist.

It will thus be seen that the objects set forth above, among those made apparent from the preceding description, are efficiently attained and, since certain changes may be made in carrying out the above methods and in the devices as set forth without departing from the spirit and scope of the invention, it is intended that all matter contained in the above description shall be interpreted as illustrative and not in a limiting sense.

What is claimed is:

1. A method of preparing a hair color composition for a person comprising the steps of:
determining the person's approximate ratio of pigmented to unpigmented hair;
determining the person's desired color level and tone;
using a palette of stock hair color formulae to select a formula to match the person's desired color and tone; and
mixing the selected formula with a developer to form the hair color composition, wherein the palette of stock hair color formulae comprises at least two tonal groups associated with the ratio of pigmented to unpigmented hair.

2. The method of claim 1, wherein the palette of stock hair color formulae comprises three tonal groups.

3. The method of claim 2, wherein the three tonal groups comprise a natural group, a bright/natural group, and a bright/cool group.

4. The method of claim 3, wherein the natural group is suitable for a person having up to 100% unpigmented hair.

5. The method of claim 3, wherein the bright/natural group is suitable for a person having about 50% unpigmented hair.

6. The method of claim 3, wherein the bright/cool group is suitable for a person having about 25% unpigmented hair.

7. The method of claim 3, wherein the natural group formula is selected from the group consisting of neutrals, brown/blonde gold, brown/blonde copper, brown/red, and brown/red violet.

8. The method of claim 3, wherein the bright/natural group formula is selected from the group consisting of gold brown/blonde, gold copper brown/blonde, copper brown/blonde, red copper brown/blonde, red brown; and red violet brown.

9. The method of claim 3, wherein the bright/cool group formula is selected from the group consisting of gold copper, copper, copper copper, red copper, red, and red red.

10. The method of claim 3, wherein the bright/cool group formula comprises ash brown/blonde.

11. The method of claim 1, wherein the palette of stock hair color formulae further comprises a calibrated level system.

12. The method of claim 1, wherein determining the ratio of pigmented to unpigmented hair comprises the use of a blend finder.

13. The method of claim 1, wherein the developer is selected from the group consisting of a 10 volume developer, a 20 volume developer, a 30 volume developer, and a 40 volume developer.

14. A personalized hair color composition formed according to the method of claim 1.

15. A personalized hair color composition formed according to the method of claim 1, wherein the composition is in the form of a creme gel.
